(19) United States
(12) Patent Application Publication
(10) Pub. No.: US 2024/0180696 A1
(43) Pub. Date: Jun. 6, 2024

(54) PROSTHETIC HEART VALVE

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Elena Sherman, Pardes Hana (IL); Anatoly Dvorsky, Haifa (IL); Eran Grosu, Kfar-Saba (IL)

(21) Appl. No.: 18/402,589

(22) Filed: Jan. 2, 2024

Related U.S. Application Data
(63) Continuation of application No. PCT/US22/36391, filed on Jul. 7, 2022.
(60) Provisional application No. 63/224,534, filed on Jul. 22, 2021.

(51) Int. Cl. A61F 2/24

(52) U.S. Cl. CPC A61F 2/2418; A61F 2/243; A61F 2/2436

(57) ABSTRACT

A prosthetic valve includes an annular frame and a valvular structure mounted within the annular frame. The valvular structure comprises a plurality of leaflets defining a plurality of commissures coupled to the annular frame. Each leaflet includes a leaflet free edge that is offset in a downstream direction from the commissures by side edges that are not attached to the frame. A material portion of the leaflet disposed between the side edges contributes a material slack that extends a radial reach of the leaflet free edge for coaptation. The prosthetic valve can be releasably coupled to a delivery device to form a delivery apparatus. The prosthetic valve can be delivered to a selected implantation site by the delivery apparatus and released at the implantation site by a handle of the delivery device.

### CROSS REFERENCE TO RELATED APPLICATIONS

This application is a continuation of PCT Application No. PCT/US2022/036391, filed Jul. 7, 2022, which claims the benefit of U.S. Provisional Patent Application No. 63/224,534, filed Jul. 22, 2021. The prior applications are incorporated by reference herein in their entireties.

### FIELD

The present disclosure relates to valvular structures and frames for prosthetic valves and to delivery apparatus and methods for implanting prosthetic valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

The prosthetic valve can include a plurality of leaflets that cycle between closed and open states during the diastolic and systolic phases of the heart. The leaflets should desirably minimize pressure gradients across the valve when open during the systolic phase and properly coapt with each other during diastole. However, achieving this operation principle with a single leaflet design over a wide range of valve diameters (for example, 20 mm to 30 mm) has been challenging. For a given valve diameter, increasing the size of the leaflets to ensure that that the leaflets can achieve proper coaptation during diastole may result in high pressure gradients during systole as the leaflets form folds and ripples along the valve opening. On the other hand, reducing the size of the leaflets to minimize pressure gradients during systole may result in malcoaptation during diastole.

### SUMMARY

In a representative example, a prosthetic valve comprises an annular frame and a plurality of leaflets positioned within the annular frame and attached to the annular frame at a plurality of locations on the annular frame. Each of the leaflets comprises a flexible sheet having a leaflet attachment edge, a leaflet free edge in opposing relation to the leaflet attachment edge, opposite first and second sides extending in an axial direction between the leaflet attachment edge and the leaflet free edge, and an upper material portion of a select height connected to the leaflet free edge; a first primary tab and a second primary tab projecting respectively from the opposite first and second sides, the first and second primary tabs offset from the leaflet free edge by the upper material portion; and a first secondary tab and a second secondary tab connected respectively to opposite ends of the leaflet free edge, the first and second secondary tabs folded about a fold line and over the upper material portion, wherein the upper material portion contributes a material slack respectively at the opposite ends of the leaflet free edge that extends a radial reach of the leaflet free edge for coaptation.

In another representative example, a prosthetic valve comprises an annular frame having an inflow end, an outflow end, a longitudinal axis defining an axial direction, and a plurality of commissure nodes positioned along a circumference of the annular frame; and a valvular structure mounted within the annular frame. The valvular structure comprises a plurality of leaflets and a plurality of commissures that attach the plurality of leaflets to the annular frame at the plurality of commissure nodes. Each leaflet comprises a leaflet attachment edge positioned in a portion of the annular frame including the inflow end and attached to the annular frame; a leaflet free edge positioned in a portion of the annular frame including the outflow end; and an upper material portion of a select height connected to the leaflet free edge, wherein the upper material portion offsets the leaflet free edge from the plurality of commissures in an axial direction by the select height, wherein the upper material portion contributes a material slack that extends a radial reach of the leaflet free edge for coaptation.

In another representative example, a delivery apparatus comprises a delivery device comprising a handle and a prosthetic valve releasably coupled to the delivery device. The prosthetic valve comprises an annular frame and a valvular structure mounted within the annular frame. The valvular structure comprises a plurality of leaflets defining a plurality of commissures coupled to the annular frame. Each leaflet comprises a free edge that is offset in a downstream direction from the commissures by sides edges that are not attached to the frame.

In another representative example, a method comprises inserting a distal end of the delivery apparatus as described above into the vasculature of a patient; advancing the prosthetic valve at the distal end to a selected implantation site; actuating the handle to release the prosthetic valve from the delivery device; and withdrawing the delivery device from the patient with the prosthetic valve implanted at the selected implantation site.

In another representative example, a prosthetic valve comprises an annular frame and a valvular structure mounted within the annular frame. The valvular structure comprises a plurality of leaflets defining a plurality of commissures coupled to the annular frame. Each leaflet comprises a leaflet free edge that is offset in a downstream direction from the commissures by side edges that are not attached to the frame, wherein the side edges contribute a material slack respectively at the opposite ends of the leaflet free edge that extends a radial reach of the leaflet free edge for coaptation.

The foregoing general description and the following detailed description are exemplary of the invention and are intended to provide an overview or framework for understanding the nature of the invention as it is claimed. The accompanying drawings are included to provide further understanding of the invention and are incorporated in and constitute a part of the specification.

### BRIEF DESCRIPTION OF THE DRAWINGS

The following is a description of the figures in the accompanying drawings.

FIG. 1 is a perspective view of a partially assembled valvular structure including leaflets, according to one example.

FIG. 2 is a perspective view of the valvular structure of FIG. 1 with the upper tabs of the leaflets folded down.

FIG. 3 is a flattened view of one of the leaflets of the valvular structure of FIGS. 1-2.

FIG. 4 is a flattened view of the leaflet of FIG. 3 with a reinforcing strip positioned along the cusp edge of the leaflet.

FIG. 5A is a flattened view of the leaflet of FIG. 3 with upper tabs of the leaflet folded down against the lower tabs of the leaflet.

FIG. 5B is a perspective view of the leaflet shown in FIG. 5A with a portion of the upper tabs fold out to form an L-shape.

FIG. 6 is a schematic illustration of deformation of the leaflet shown in FIG. 5A during diastole.

FIG. 7A is a perspective view of one example of a prosthetic valve that incorporates leaflets of the type shown in FIGS. 1-6.

FIG. 7B is a perspective view of the prosthetic valve of FIG. 7A with an outer skirt as a sealing member.

FIG. 8A is a perspective view of a frame for a prosthetic valve.

FIG. 8B is a front portion of the frame shown in FIG. 8A.

FIG. 9A is a perspective view of a frame including angled cantilevered struts.

FIG. 9B is a perspective view of the frame of FIG. 9A with a flared skirt mounted on the outside surface of the frame as a sealing member.

FIGS. 10A-10C are schematic illustrations of a process of forming the commissures of a valvular structure.

FIG. 11 is a flattened view of an alternative leaflet for a valvular structure of a prosthetic valve.

FIG. 12 is a schematic illustration of a delivery apparatus including a delivery device attached to a prosthetic valve.

FIG. 13A is a flattened view of a leaflet, according to another example.

FIG. 13B is a flattened view of the leaflet of FIG. 13A with the upper tabs of the leaflet folded down.

FIG. 14 is an illustration from a video recording of a prosthetic valve with the leaflets of FIG. 13A in a closed state.

FIG. 15 is an illustration from a video recording of a prosthetic valve with leaflets having the leaflet geometry of FIG. 3 in a closed state.

FIG. 16A is an illustration from a video recording of a prosthetic valve with the leaflets of FIG. 13A and a valve diameter of 26 mm in an open state.

FIG. 16B is an illustration from a video recording of a prosthetic valve with the leaflets of FIG. 13A and a valve diameter of 29 mm in an open state.

FIG. 17A is an illustration from a video recording of a prosthetic valve with leaflets having the leaflet geometry of FIG. 3 and a valve diameter of 26 mm in an open state.

FIG. 17B is an illustration from a video recording of a prosthetic valve with leaflets having the leaflet geometry of FIG. 3 and a valve diameter of 29 mm in an open state.

### DETAILED DESCRIPTION

For the purposes of this description, certain specific details are set forth herein in order to provide a thorough understanding of disclosed examples. In some cases, as will be recognized by one skilled in the art, the disclosed examples may be practiced without one or more of these specific details, or may be practiced with other methods, structures, and materials not specifically disclosed herein. In some instances, well-known structures and/or processes associated with prosthetic valves and delivery apparatuses have been omitted to avoid obscuring novel and non-obvious aspects of the disclosed examples.

The disclosed examples are described via preferred implementations and examples. All the implementations and examples described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

A prosthetic valve that can be implanted within any of the native valves of the heart (for example, the aortic, mitral, tricuspid, and pulmonary valves) is described herein. Leaflets for the valvular structure of the prosthetic valve are described herein. In certain examples, the leaflet includes a leaflet free edge and tabs used to form commissures that attach the leaflet to an annular frame. The leaflet has an upper material portion that offsets the leaflet free edge from the tabs such that the leaflet free edge is not attached to the annular frame when commissures are formed at the tabs. During the diastolic and systolic phases of the heart, the leaflets cycle between an open state, where the leaflet free edges are separated from each other, and a closed state, where the leaflet free edges coapt. The leaflets can be configured such that in the open state the upper material portions protrude generally vertically relative to the commissures. During diastole, the upper material portions can be tensioned such that they extend radially inwardly and participate in leaflet coaptation by contributing a material slack that allows the leaflet free edges to extend further toward cach other and coapt with minimal or no gaps therebetween. The leaflet geometry can enable a wider range of valve sizes with proper coaptation and minimized pressure gradients across the valve.

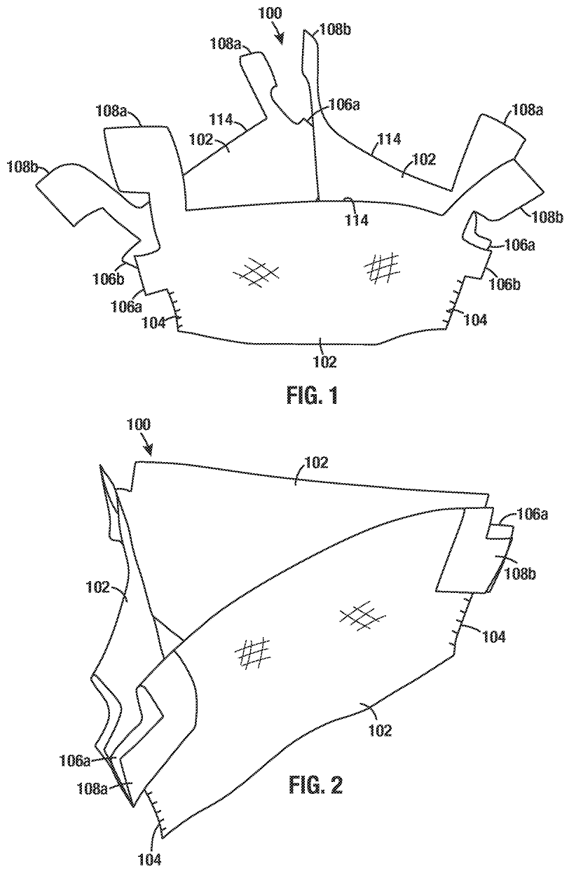

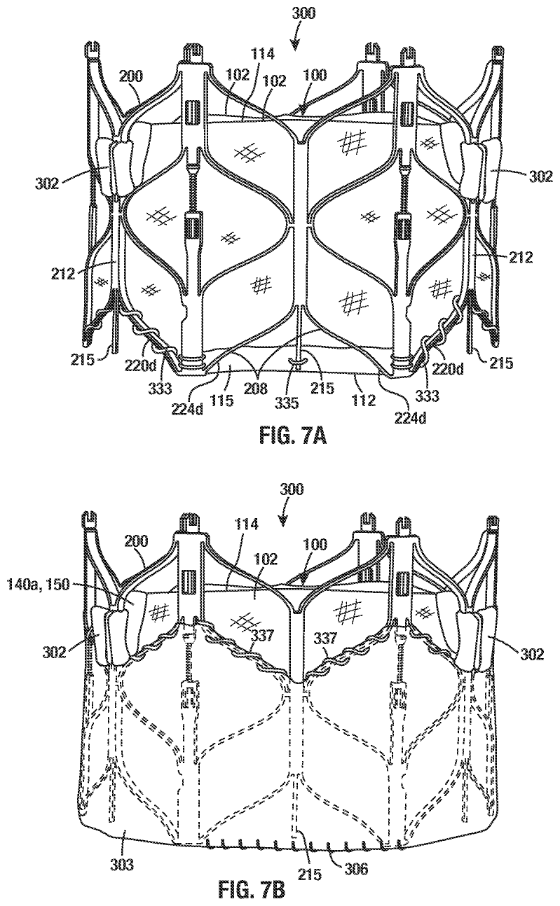

Turning now to the drawings, FIG. 1 shows an exemplary valvular structure 100 (also referred to as a leaflet assembly), in a partially assembled state. The valvular structure 100 may be mounted within an annular frame of a prosthetic valve (for example, valve 300 of FIG. 7A, discussed below). In one illustrative implementation, the valvular structure 100 includes a plurality of leaflets 102 that in use are coupled to the frame and open and close to regulate flow of blood through the frame. The leaflets 102 have leaflet free edges 114 that move radially to coapt with each other during diastole. The valvular structure 100 is shown as having three leaflets 102 that can be arranged to collapse in a tricuspid arrangement. However, in other implementations, the valvular structure 100 may have greater or fewer number of leaflets than three (for example, one or more leaflets). The leaflets 102 are made of a flexible material. In a particular example, the leaflets 102 may be made in whole or in part from pericardial tissue (for example, bovine pericardial tissue), biocompatible synthetic materials, or various other suitable natural or synthetic materials as known in the art and described in, for example, U.S. Pat. No. 6,730,118, the relevant disclosure of which is incorporated herein by reference.

In the partially assembled state shown in FIG. 1, the leaflets 102 may be partially secured together at a portion of their adjacent sides by sutures 104. Suturing of adjacent sides as shown at 104 may occur before or after positioning the leaflets within a frame. Each leaflet 102 includes primary tabs 106a, 106b (which are the lower tabs before folding, as described below) at opposite sides of the leaflet and secondary tabs 108a, 108b (which are the upper tabs before folding) at an outflow end of the leaflet where the leaflet free edge 114 is located. As shown in FIG. 2, the secondary tabs 108a, 108b can be folded down and positioned adjacent to the primary tabs 106a, 106b. In this position, the secondary tabs 108a, 108b and the respective primary tabs 106a, 106b can cooperate to form commissures at the adjacent sides of the leaflets 102. These commissures can be mounted within a frame and in a manner to secure the leaflets to the frame by the commissures. The leaflet 102 has an upper material portion that offsets the leaflet free edge 114 from the primary tabs 106a, 106b. As will be explained further, the secondary tabs 108a, 108b have portions that can overlap the upper material portion in order to position the secondary tabs 108a, 108b adjacent to the primary tabs 106a, 106b. The portions of the secondary tabs 108a, 108b that overlap the upper material portion do not participate in forming commissures and are not attached to the frame.

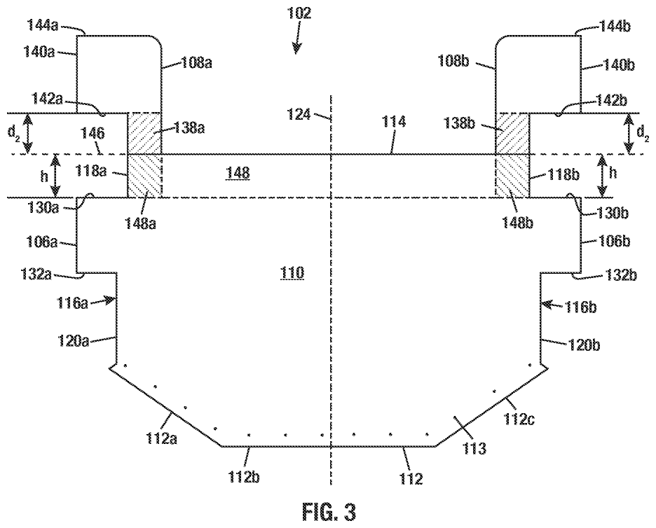

Referring to FIG. 3, in one illustrative implementation, a leaflet 102 includes a flexible sheet 110 forming a leaflet body. The flexible sheet 110 can comprise any of the example leaflet materials previously mentioned or known in the art. In a non-limiting example, the thickness of the flexible sheet 110 can be in a range from 0.1 mm to 1 mm. The flexible sheet 110 includes a leaflet attachment edge 112 (lower edge in the figure) and a leaflet free edge 114 (upper edge in the figure) that is in opposing relation to the leaflet attachment edge 112 (also referred to as the inlet edge or cusp edge of the leaflet). When the leaflet 102 is mounted within an annular frame as part of a valvular structure, the leaflet attachment edge 112 can be positioned in an inflow end portion of the frame, and the leaflet free edge 114 can be positioned in an outflow end portion of the frame. The terms “inflow” and “outflow” are related to the normal direction of flow through the frame.

The leaflet attachment edge 112 may have a truncated V-shape or tapered shape as shown. In one example, the leaflet attachment edge 112 may include linear edges 112a, 112b, 112c that are arranged to form the truncated V-shape or tapered shape. In other examples, the leaflet attachment edge 112 may have a truncated V-shape or tapered shape composed of one or more curved edges or a combination of linear edges and curved edges. The leaflet free edge 114 may be a linear edge as shown or an approximately linear edge. The approximately linear edge may be formed by two or more linear edges with different inclination angles or by a curved edge with a slight curvature. In other implementations, the leaflet attachment edge 112 may have a scalloped shape.

The flexible sheet 110 in the illustrated example includes opposite sides 116a, 116b that extend axially between respective ends of the leaflet attachment edge 112 and the leaflet free edge 114. The axial direction of the leaflet 102 is indicated by longitudinal axis 124, which may be an axis that is transverse to the leaflet attachment edge 112 and the leaflet free edge 114. The longitudinal axis 124 may be an axis about which leaflet 102 is symmetrical. The side 116a includes side edges 118a, 120a, which are spaced apart in the axial direction and are generally parallel to the longitudinal axis 124.

The side 116b includes side edges 118b, 120b, which are spaced apart in the axial direction and are generally parallel to the longitudinal axis 124. The side edges 120a, 120b (also referred to as sub-commissure edges of the leaflet) are connected to opposite ends of the leaflet attachment edge 112. During assembly of the valvular structure, the side edges 120a, 120b may be sutured to side edges 120b, 120a of adjacent leaflets (as shown at 104 in FIG. 1) or attached to a frame. However, in particular examples, the side edges 118a, 118b are not sutured to side edges 118a, 118b of adjacent leaflets nor attached to a frame. Instead, the side edges 118a, 118b can form part of an upper material portion 148 of the leaflet that offsets the leaflet free edge 114 from the commissure forming tabs.

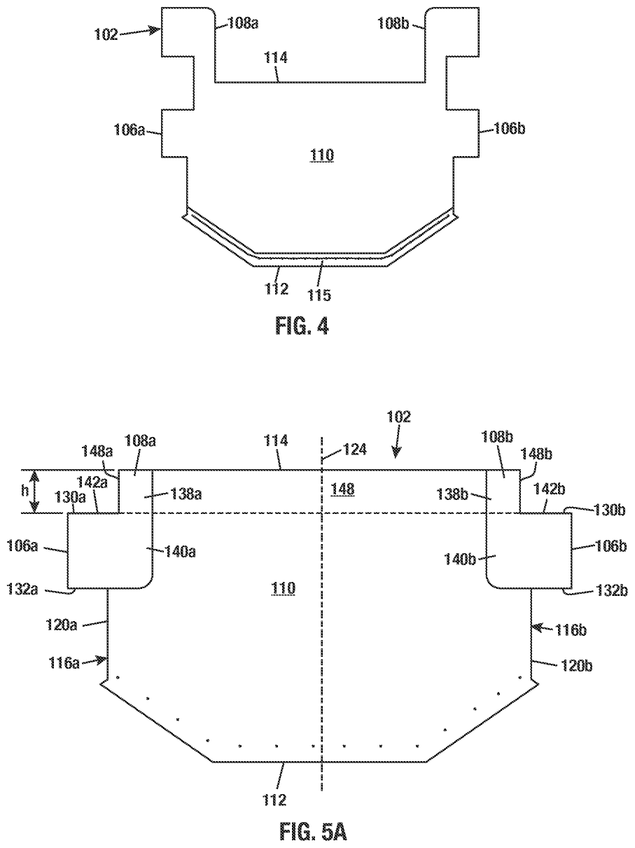

The leaflet 102 may be attached to a frame (for example, to struts of the frame) along the leaflet attachment edge 112. To protect the leaflet material in this attachment area from tears, a reinforcement material may be provided in the area. For illustration purposes, FIG. 4 shows a reinforcing strip 115 attached to a portion of the flexible sheet 110 adjacent to the leaflet attachment edge 112. The reinforcing strip 115 is preferably made of a tear resistant material that is biocompatible. In one example, the tear resistant material may be polyethylene terephthalate (PET), although various other synthetic or natural materials may be used.

In general, it is desired to strengthen the flexible sheet 110 at the leaflet attachment edge 112 without adding too much bulk to the edge. As an example, the thickness of the reinforcing strip 115 may be less than 6 mil (0.15 mm), preferably less than 4 mil (0.1 mm), and more preferably less than 2 mil (0.05 mm). In one example, as shown in FIG. 3, the flexible sheet 110 may have pre-formed holes 113 adjacent to the leaflet attachment edge 112 to facilitate attachment of the reinforcing strip to the flexible sheet 110 by stitching, although any other suitable method of attaching the reinforcing strip to the flexible sheet 110 may be used.

The leaflet 102 has opposite primary tabs 106a, 106b (lower tabs in the figure) projecting from opposite sides 116a, 116b of the flexible sheet 110. The primary tabs 106a, 106b are involved in forming commissures of the valvular structure as previously described. The primary tab 106a extends between the side edges 118a, 120a. The primary tab 106b extends between the side edges 118b, 120b. The primary tabs 106a, 106b have first edges 130a, 130b (top edges in the figure) and second edges 132a, 132b (bottom edges in the figure). Each of the first edges 130a, 130b is axially offset (that is, offset in the axial direction of the leaflet) from the leaflet free edge 114 by the upper material portion 148 of the flexible sheet 110 that extends between the side edges 118a, 118b. The upper material portion 148 has a height h>0 that determines the offset distance of the leaflet free edge 114 from the primary tabs. In some cases, the height h can be greater than a thickness of the flexible sheet 110.

The leaflet 102 has secondary tabs 108a, 108b (upper tabs in the figure) at opposite ends of the leaflet free edge 114. The secondary tab 108a has a first tab portion 138a and a second tab portion 140a arranged orthogonally to form an L shape. Similarly, the secondary tab 108b has a first tab portion 138b and a second tab portion 140b arranged orthogonally to form an L shape. The first tab portion 138a extends from one end of the leaflet free edge 114 to a top end of the side edge 118a. The first tab portion 138b extends from another end of leaflet free edge 114 to a top end of the side edge 118b. The second tab portions 140a, 140b are offset from the leaflet free edge 114 in the axial direction by the length (or height) of first tab portions 138a, 138b.

The secondary tab 108a is located on the same side of the leaflet 102 as the primary tab 106a (left side in the figure) and forms a cooperating pair with the primary tab 106a, which means that tabs 108a, 106a will work together to form a commissure with an adjacent pair of primary and secondary tabs of an adjacent leaflet. The secondary tab 108b is located on the same side of the leaflet 102 as the primary tab 106b (right side in the figure) and forms a cooperating pair with the primary tab 106b, which means that tabs 108b, 106b will work together to form a commissure with an adjacent pair of primary and secondary tabs of an adjacent leaflet.

The second tab portions 140a, 140b of the secondary tabs 108a, 108b have first edges 142a, 142b (bottom edges in the figure) and second edges 144a, 144b (top edges in the figure). Each of the first edges 142a, 142b is axially offset (that is, offset in the axial direction of the leaflet) from leaflet free edge 114 by an offset distance d₂>0 (that is, the length or height of the first tab portions 138a, 138b). The offset distance d₂ can be the same or substantially the same as the height h of the upper material portion 148.

When forming the valvular structure, the secondary tabs 108a, 108b can be folded about a horizontal fold line 146 to place the second tab portion 140a against or adjacent to the primary tab 106a and the second tab portion 140b against or adjacent to the primary tab 106b. In one example, the fold line 146 is coincident or aligned with the leaflet free edge 114, as shown in FIG. 3. In some cases, the second tab portions 140a, 140b may be folded against the primary tabs 106a, 106b by matching the first edges 142a, 142b of the second tab portions 140a, 140b with the first edges 130a, 130b of the primary tabs 106a, 106b, respectively.

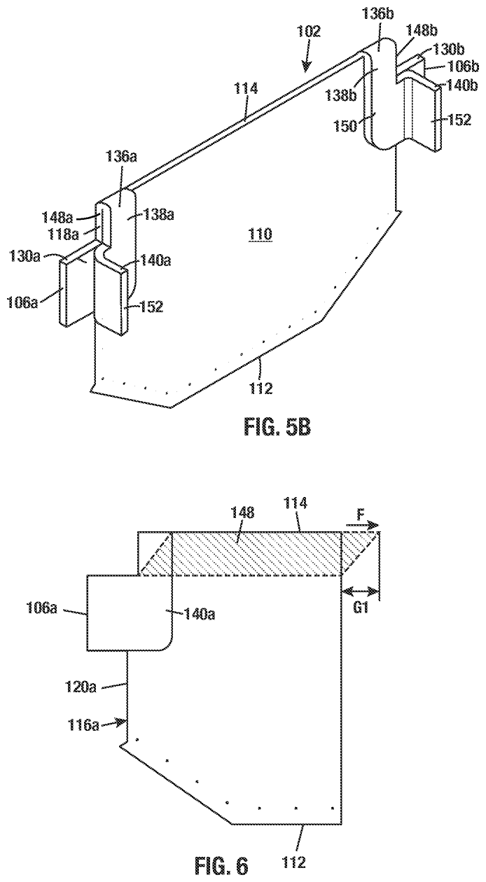

As shown in FIGS. 5A and 5B, when the second tab portions 140a, 140b are placed against or adjacent to the primary tabs 106a, 106b, the first tab portion 138a is folded against a corner portion 148a (indicated in FIG. 3) of the upper material portion 148 of the flexible sheet. Similarly, the first tab portion 138b is folded against a corner portion 148b (indicated in FIG. 3) of the upper material portion 148 of the flexible sheet. As such, the first tab portions 138a, 138b and the upper material portion 148 protrude relative to the primary tabs 106a, 106b.

In an assembled state (that is, when the leaflets are part of a valvular structure mounted within a frame), the upper material portion 148 of the leaflet extends relatively vertically from the commissures in the open state (for example, during systole). The side edges 118a, 118b of the upper material portion 148 and the first tab portions 138a, 138b of the secondary tabs 108a, 108b are not attached to the frame, which means that the upper material portion 148 and the first tab portions 138a, 138b are able to deflect with the leaflet free edge 114 when the leaflet free edge 114 moves radially to coapt with the leaflet free edges of other leaflets.

The upper material portion 148 provides a material slack adjacent to the leaflet free edge 114 that extends the radial reach of the leaflet free edge 114 during coaptation. FIG. 6 illustrates the deformation of the upper material portion 148 that occurs during diastole. The non-retained upper material portion 148 is able to move radially inward (that is, towards the middle of the valve) in response to tension applied to the leaflet (indicated as F) during diastole and thus provides additional material for coaptation (identified as gain G1).

The amount of material slack provided by the upper material portion 148 depends on the height h (indicated in FIGS. 3 and 5A) of the upper material portion 148, that is, the amount by which the upper material portion 148 protrudes relative to the primary tabs 106a, 106b (or relative to commissures formed at primary tabs 106a, 106b when the leaflet is mounted in a frame as part of a valvular structure).

In the design of the leaflet 102, the height h of the upper material portion 148 can be selected to provide the material slack to achieve complete leaflet coaptation. In one example, for valve diameters in a range from 20 mm to 30 mm and using three leaflets, the height h can be in a range from 0.5 mm to 5 mm. In a particular example, the height h in a range from 0.5 mm to 2 mm has been found to be effective for valve diameters in a range from 26 mm to 29 mm.

Since the upper material portion 148 can act to extend the reach of the leaflet for coaptation, it is possible to shorten the overall height of the leaflet and narrow the width of the leaflet to enable smaller valve sizes that meet desired coaptation and pressure gradient performance. Moreover, shortening the leaflets allows the free edges 114 of the leaflets to be spaced upstream of the outflow end of the frame of the prosthetic valve (see, for example, frame 200 of FIGS. 7A-7B), which minimizes the risk of blocking the coronary ostia and can help preserve access to the coronary ostia during a subsequent valve-in-valve procedure.

FIG. 7A shows an exemplary prosthetic valve 300 including the valvular structure 100 with the leaflets 102 mounted within and to an annular frame 200. The valvular structure 100 is shown in a fully assembled state with commissures 302 that couple the leaflets 102 to the frame 200. The leaflets 102 are also coupled to the frame 200 along their leaflet attachment edges 112 (the lower edges in the figure; also referred to as “cusp edges”). Due to shaping of the leaflet attachment edges 112 and the manner in which the leaflet attachment edges 112 are attached to the frame 200, the lower edge of the valvular structure 100 is coupled to the frame 200 along a generally scallop shaped attached line.

The prosthetic valve 300 may include one or more skirts mounted around the frame 200. For example, as shown in FIG. 7B, the prosthetic valve 300 may include an outer skirt 303 mounted around an outer surface of the frame 200. The outer skirt 303 functions as a sealing member for the prosthetic valve 300 by sealing against the tissue of the native valve annulus and helping to reduce paravalvular leakage past the prosthetic valve. In some cases, an inner skirt may be mounted around an inner surface of the frame 200. The inner skirt can function as a sealing member to prevent or decrease perivalvular leakage, to anchor the leaflets to the frame, and/or to prevent the leaflets against damage caused by contact with the frame during crimping and during working cycles of the prosthetic valve. The inner and outer skirts can be formed from any of various suitable biocompatible materials, including any of various synthetic materials, including fabrics (for example, PET fabric) or natural tissue (for example, pericardial tissue). Further details regarding the use of skirts or scaling members in prosthetic valve can be found, for example, in U.S. Patent Application No. 62/854,702, the relevant disclosure of which is incorporated herein by reference.

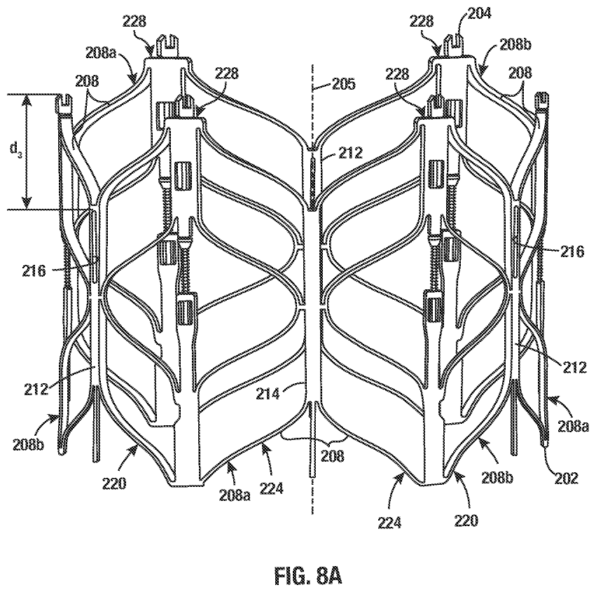

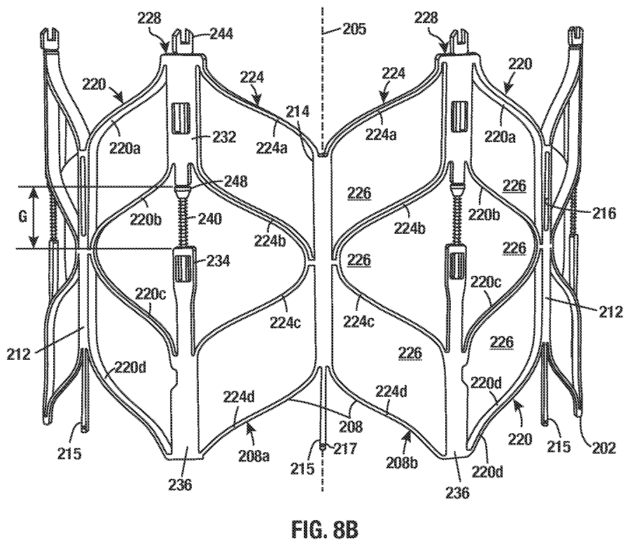

FIGS. 8A and 8B show one illustrative implementation of the annular frame 200. However, it should be understood that the valvular structure 100 and the frame 200 can be used independently of each other in various prosthetic valve designs. The frame 200 has an inflow end 202 (bottom end in the figure) and an outflow end 204 (top end in the figure). The axial direction of the frame 200 is indicated by a longitudinal axis 205, which extends from the inflow end 202 to the outflow end 204. The frame 200 includes a plurality of frame sections 208 extending between the inflow end 202 and the outflow end 204. Axially extending main frame posts 212 are disposed between adjacent frame sections 208 and interconnect frame sections 208 around the circumference of the frame to form the annular shape of the frame. In the illustrated implementation, the frame 200 has three frame sections 208, each frame section 208 extending between two main frame posts 212. In other implementations, the frame 200 may have fewer or greater than three frame sections 208. Each leaflet of the valvular structure can extend over one of the frame sections 208.

Each frame section 208 has two frame subsections 208a, 208b interconnected by an axially extending auxiliary frame post 214. Each frame section 208 may have a reflection symmetry about the respective auxiliary frame post 214. Each frame subsection 208a, 208b extends between a main frame post 212 and an auxiliary frame post 214 and is connected on one side to the adjacent main frame post 212 and on the opposite side to the adjacent auxiliary frame post 214. Each of the frame subsections 208a, 208b may have an actuator portion 228 that can be used to radially expand the frame.

As shown more clearly in FIG. 8B, each frame subsection 208a, 208b includes struts 220a, 220b, 220c, 220d (collectively, struts 220) and struts 224a, 224b, 224c, 224d (collectively, struts 224). The numbers of the struts 220, 224 illustrated are not intended to be limiting. The struts 220a, 220b, 220c, 220d are spaced apart and aligned in the axial direction in one half of the respective frame subsection 208a, 208b and form one vertical column of cells 226. The struts 224a, 224b, 224c, 224d are spaced apart and aligned in the axial direction in the other half of the respective frame subsection 208a, 208b and form another vertical column of cells 226. The struts 220, 224 are arranged in pairs of struts spaced apart from each other in the axial direction of the frame, for example, strut pair 220a and 224a; strut pair 220b and 224b; strut pair 220c and 224c; and strut pair 220d and 224d. Struts 220, 224 are desirably (but not necessarily) curved, forming cells 226 with curved edges. The curved geometry formed by the upper struts 220a, 224a, 220b, 224b is inverted relative to the curved geometry formed by the lower struts 220c, 224c, 220d, 224d. The curved geometries lend flexibility to the frame subsections 208a, 208b.

In each frame subsection 208a, 208b, first ends of the struts 220 are connected to an adjacent main frame post 212, while first ends of the struts 224 are connected to an adjacent auxiliary frame post 214. Second ends of the struts 220, 224 may be connected to the actuator portion 228 disposed in the frame subsection.

In one example, each actuator portion 228 includes an upper support arm 232 and a lower support arm 236, which are longitudinally aligned and separated by a gap G. An adjustable link is formed between the support arms 232, 236 to allow the size of gap G to be adjustable. The adjustable link may include a bore inside the upper support arm 232, a threaded nut 234 at an upper end of the lower support arm 236, and a threaded rod (or screw) 240 that extends through the bore of the upper support arm 232, through gap G, into the threaded nut 234 at the upper end of the lower support arm 236. A head 244 of the threaded rod 240 is accessible from an upper end of the upper support arm 232 and can be operated to rotate the threaded rod 240 and cause relative motion between the support arms 232, 236, thereby adjusting the size of gap G and producing radial expansion and compression of the frame, as further described below.

The second ends of the upper struts 220a, 224a, 220b, 224b are connected to the upper support arm 232, and the second ends of the lower struts 220c, 224c, 220d, 224d are connected to the lower support arm 236. As a result, the struts 220, 224 can deflect as the threaded rod 240 is rotated and the size of gap G is adjusted. As shown in the illustrated example, the nut 234 can be an integral upper portion of the lower support arm 236, which has internal threads that engage the outer threads of the rod 240. In alternative examples, the nut 234 can be a separate component that is housed within an upper portion of the lower support arm 236. The head 244 of each threaded rod 240 can be releasably coupled to a respective actuator of a delivery apparatus to produce rotation of the threaded rods 240, as further described below.

The actuator portions 228 can be used to radially expand and lock the frame 200 in a desired working diameter inside the patient's body. In one example, the lower support arm 236 may be held steady while the threaded rod 240 is rotated, for example, via an external driver of a prosthetic valve delivery apparatus that engages the head 244 of the threaded rod 240. The threaded rod 240 may be rotated in a first direction to move the upper support arm 232 towards the lower support arm 236 and thereby decrease the size of gap G, which can have the effect of radially expanding the frame 200. Alternatively, the lower support arm 236 may be held steady while the threaded rod 240 is rotated in a second direction to move the upper support arm 232 away from the lower support arm 236 and thereby increase the size of gap G, which can have the effect of radially compressing the frame 200. In some cases, a stopper, such as a nut 248, may be installed on the threaded rod 240 to limit the travel of the threaded rod 240 while rotating the threaded rod 240 to radially compress the frame 200. For example, the nut 248 may be arranged to abut a lower end of the upper support arm 232 and stop further motion of the threaded rod 240 in order to prevent over-crimping of the frame 200.

In an alternative implementation, the upper support arm 232 and the lower support arm 236 of each actuator portion 228 can be threaded, and the threaded rod 240 can be provided with threaded end portions to threadedly engage the support arms 232, 236. The threaded end portions can by oppositely threaded such that rotation of the threaded rod will move the supports arms toward or away from each other, depending on the direction of rotation.

The struts 220, 224 provide attachment points for leaflets and skirts(s) of a prosthetic valve. As an example, selected ones of the lowermost struts 220d, 224d may serve as attachment points for leaflet attachment edges as well as inflow edges of skirt(s). As shown in FIG. 7A, the lowermost struts 220d, 224d connected to edge sections 112a, 112c (in FIG. 3) form a generally scallop shape attachment line. The leaflets can track this scallop line to form the scallop geometry at the lower edge of the valvular structure. The remaining struts, that is, other than the lowermost struts, may selectively serve as attachment points for other parts of the valve, such as the skirt(s).

In one example, the frame 200 may include additional struts that provide attachment points for leaflets and skirts(s). As an example, as shown in FIG. 8B, cantilevered struts 215 may be attached to the lower ends of the main frame posts 212 and the auxiliary frame posts 214 and may extend (downwardly in the figure) from the end of the respective frame post to the inflow end 202 of the frame 200. Each cantilevered strut 215 may have a hole or eye 217 or other features to receive a suture or other attachment material for connecting an adjacent edge section 112b (in FIG. 3) of a leaflet, the outer skirt 303 (in FIG. 7B), and/or an inner skirt.

In the example shown in FIGS. 8A and 8B, the cantilevered struts 215 extend vertically (that is, in the axial direction) from the frame posts 212, 214. FIG. 9A shows an alternative frame implementation 200′ where cantilevered struts 215′ are attached to the frame posts 212, 214 and are angled radially outward relative to the axial direction in a shape-set, biased state. When a skirt is mounted around the outer surface of frame 200′, the angled cantilevered struts 215′ can push the skirt radially outward, creating a flared skirt, as illustrated at 303′ in FIG. 9B. The flared skirt as a sealing member may better conform to the native valve geometry.

The angled cantilevered struts 215′ may comprise a flexible and/or shape memory material such as Nitinol that naturally biases the cantilevered struts radially outward when the prosthetic valve is released from a delivery apparatus. When the prosthetic valve is in a radially compressed, delivery state, the cantilevered struts 215′ can be compressed radially inwardly such that the entire frame 200′ can assume a cylindrical shape having a constant diameter from its inflow end to its outflow end. The free ends of the angled cantilevered struts 215′ may have holes or eyes or other features to receive a suture or other attachment material for connecting the inflow edge of the skirt 303′ to the struts 215′. The alternative frame implementation 200′ with the angled cantilevered struts 215′ may be used in the prosthetic valve 300 shown in FIGS. 7A and 7B in the same way as shown for frame 200.

Returning to FIGS. 8A and 8B, the main frame posts 212 have commissure nodes, which are sites where commissures will be formed to couple the leaflets to the frame 200. In one example, the commissure nodes are provided by openings 216 formed in the main frame posts 212. The openings 216 are sized to receive leaflet tabs that form the commissures (302 in FIGS. 7A and 7B). Hereafter, the openings 216 are referred to as commissure windows. The commissure windows 216 are spaced apart about the circumference of frame 200 (or angularly spaced apart about frame 200). The spacing may or may not be even.

In one example, the commissure windows 216 are axially offset from the outflow end 204 of the frame 200 by an offset distance d₃ (indicated in FIG. 8A). As an example, the offset distance d₃ may be in a range from 2 mm to 6 mm. In general, the offset distance d₃ should be selected such that when the leaflets with the free upper material portions are attached to the frame 200 via the commissure windows 216, the free upper material portions will not protrude from the outflow end 204 of the frame 200. Thus, the offset distance d₃ can be selected to be greater than the height of the upper material portions in the open state of the leaflets.

The various struts, posts, and support arms in the frame 200 may be made of any of various suitable materials, such as stainless steel, a cobalt aluminum alloy, or a nickel titanium alloy (for example, Nitinol). In particular examples, the frame 200 can be cut (for example, laser cut) or otherwise formed from a tubular piece of material to form all components of the frame, except for threaded rods 240, which can be separately formed and assembled to the fully formed frame. In other examples, the frame 200 may be constructed by forming individual components and then mechanically assembling and connecting the individual components together, such as by welding individual frame components to each other or by connecting frame components to each other with pivot connectors (for example, rivets) to form hinges at the junctions between struts. Examples of constructing a prosthetic valve frame from individual formed components connected at hinges are described in U.S. Patent Publication Nos. 2018/0153689 and 2018/0344456, and U.S. patent application Ser. Nos. 16/105,353 and 62/748,284, the relevant disclosures of which are incorporated herein.

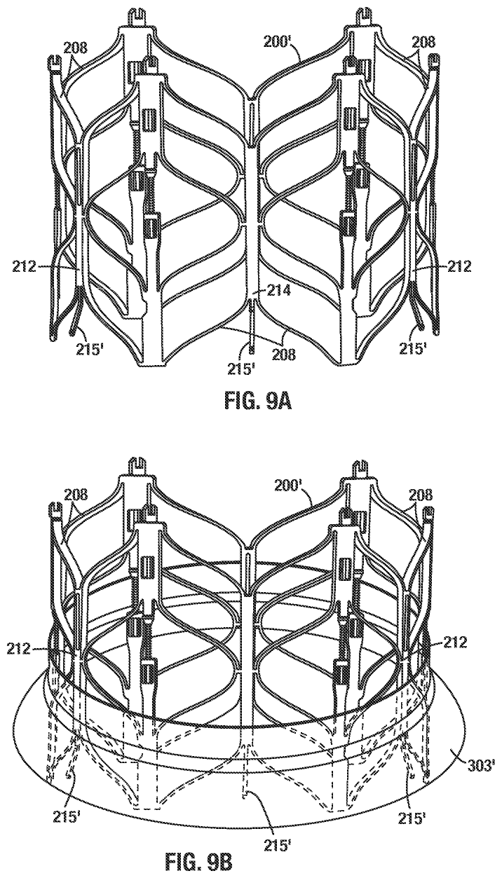

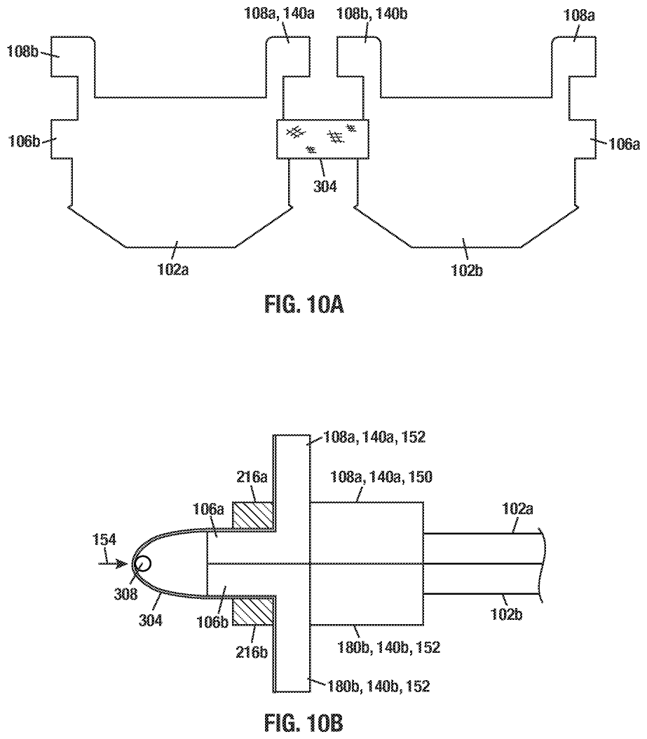

Referring to FIG. 10A, in particular examples, a leaflet assembly or valvular structure can be formed by connecting a flexible connector 304 to a pair of leaflets 102a, 102b at a primary tab 106a of the leaflet 102a and a primary tab 106b of the leaflet 102b. The flexible connector 304 can be connected to the primary tabs 106a, 106b with sutures. The flexible connector 304 can comprise, for example, a piece of fabric (for example, PET fabric). A wedge element 308 (FIGS. 10B and 10C) can be connected to one side of the flexible connector 304. The wedge element 308 can comprise, for example, a relatively heavy gauge suture, such as a braided suture (for example, an ETHIBOND suture), or a piece of fabric. At this stage, a reinforcing strip 115 can be connected to the leaflet attachment edge 112 of each leaflet 102a, 102b (as shown in FIGS. 4 and 7A).

A third leaflet 102 (not shown in FIG. 10A; desirably already having a respective reinforcing strip 115) can be similarly coupled to leaflets 102a, 102b by connecting a second connector 304 to the primary tab 106b of the leaflet 102a and to a primary tab of the third leaflet and connecting a third connector 304 to the primary tab 106a of the leaflet 102b and to the other primary tab of the third leaflet, thereby forming a leaflet assembly of three leaflets (similar to the assembly shown in FIG. 2) coupled to each other with respective connectors 304. It should be understood that the leaflet assembly can include additional leaflets coupled to each other with additional connectors 304.

The adjacent sub-commissure edges 120a, 120b (in FIG. 3) of adjacent leaflets can be connected to each other with sutures 104, as shown in FIGS. 1 and 2. The sutures can, for example, form in-and-out stitches or whip stitches that extend through a pair of adjacent sub-commissure edges 120a, 120b.

The secondary tabs 108a, 108b of each leaflet 102 can then be folded downwardly against their corresponding primary tabs 106a, 106b. For example, referring to FIG. 10A, the secondary tab 108a of the leaflet 102a can be folded downwardly against the primary tab 106a of the leaflet 102a on the same side of the leaflet as the connector 304. In this manner, the second tab portion 140a of the secondary tab 108a can partially overlap a portion of the connector 304 (that is, a portion of the connector 304 is situated between the primary tab 106a and the second tab portion 140a). Similarly, the secondary tab 108b of the leaflet 102b can be folded downwardly against the primary tab 106b of the leaflet 102b.

After folding the secondary tabs 108a, 108b, each of the second tab portions 140a, 140b can be folded lengthwise along a vertical fold axis to form an L-shape having an inner portion 150 and an outer portion 152 (see FIG. 5B). The inner portion 150 can contact the inner surface of the leaflet and the outer portion 152 can contact the connector 304. The outer portions 152 can be sutured to the connector 304, such as with sutures 310 (shown in FIG. 10C)

Referring now to FIG. 10B, a commissure tab assembly formed by a connector 304, the tabs 106a, 108a of the leaflet 102a, and the tabs 106b, 108b of the leaflet 102b can be coupled to a commissure window 216 of a frame as follows. As shown in FIG. 10B, the connector 304 and the primary tabs 106a, 106b can be inserted through a commissure window 216 defined by a pair of struts 216a, 216b (from the inside of the frame to the outside of the frame), while the secondary tabs 108a, 108b remain inside the frame. The commissure tab assembly is then pressed inwardly at the wedge element 308 (in the direction of arrow 154) such that the outer portion 152 of the secondary tab 140a and a portion of the connector 304 are against the frame on one side of the window 216 and the outer portion 152 of the secondary tab 140b and a portion of the connector 304 are against the frame on the other side of the window 216.

As shown in FIG. 10C, the pressing of the commissure tab assembly also causes the primary tab 106a and a portion of the connector 304 to fold around the strut 216a on the outside of the frame opposite the outer portion 152 of the secondary tab 108a, and the primary tab 106b and a portion of the connector 304 to fold around the strut 216b on the outside of the frame opposite the outer portion 152 of the secondary tab 108b. A pair of suture lines 312 can be formed to retain the primary tabs 106a, 106b against the frame. Each suture line 312 extends through the connector 304, a primary tab, the wedge element 308, and another portion of the connector 304.

Each primary tab 106a, 106b can be secured to a corresponding secondary tab 108a, 108b with a primary suture line 314. Each suture line 314 extends through one layer of the connector 304, a primary tab 106a, 106b, another layer of the connector 304, another layer of the connector 304, and the outer portion 152 of the secondary tab 108a, 108b. The end portions of the suture material used to form the primary suture lines 314 (or separate sutures) can be used to form whip stitches 316 at the adjacent outer edges of the tabs 106a, 108a and at the adjacent outer edges of the tabs 106b, 108b. A first set of stitches 316 can extend through the tabs 106a, 108a and two layers of the connector 304 between the tabs 106a, 108a, and a second set of stitches can extend through the tabs 106b, 108b and two layers of the connector 304 between the tabs 106b, 108b.

During valve cycling, the leaflets 102a, 102b can articulate primarily at inner edges 156 of the folded inner portions 150. However, when the prosthetic valve is radially compressed to a delivery state, the relatively higher forces acting on the leaflets can cause the leaflets to splay apart about a longitudinal axis 158, allowing for a smaller crimped diameter.

The remaining commissure tab assemblies of the leaflet assembly can be coupled to respective commissure windows 216 of the frame 200 in the same manner as described above. Further details of the method for forming the commissure tab assemblies and coupling them to the frame are disclosed in U.S. Pat. No. 9,393,110, which is incorporated herein by reference. It should be noted that FIGS. 10A-10C show one exemplary technique for coupling the commissures of a leaflet assembly to a frame. Other techniques, methods, and mechanisms can be used for coupling the commissures of the leaflet assembly to the frame 200, such as any of those disclosed in U.S. Pat. No. 9,393,110, U.S. Publication No. 2018/0325665, or U.S. Application No. 63/003,085, filed Mar. 31, 2020, which are incorporated herein by reference.

FIG. 7A shows an example of attaching a leaflet attachment edge 112 of a leaflet 102 to a frame section 208 of the frame 200. Inclined portions of leaflet attachment edge 112 (shown as 112a, 112c in FIG. 3) are attached to the lowermost struts 220d. The horizontal portion of leaflet attachment edge 112 (shown as 112b in FIG. 3) extends between the lowermost struts 220d of adjacent frame subsections and optionally can be attached in the middle to an adjacent cantilevered strut 215. The leaflet attachment edge 112 and the reinforcing strip 115 may be attached to the struts 220d with sutures 333. A suture 335 can be used to attach the leaflet attachment edge 112 and the reinforcing strip 115 to an adjacent strut 215.

After the commissures 302 have been formed and the leaflet attachment edges 112 have been secured to the frame, a skirt 303 can be mounted around the outer surface of frame 200 as shown in FIG. 7B. In one example, the inflow edge of the skirt 303 (lower edge in the figure) can be attached to the leaflet attachment edges that have already been secured to frame 200 as well as to the cantilevered struts 215 of the frame as shown by sutures 306. The outflow edge of the skirt 303 (the upper edge in the figure) can be attached to selected struts with sutures 337. In implementations where the prosthetic valve includes an inner skirt, the inflow edge of the inner skirt can be secured to the leaflet attachment edges before securing the leaflet attachment edges to the frame so that the inner skirt will be between the leaflets and the inner surface of the frame. After the inner skirt and leaflets are secured in place, then the outer skirt can be mounted around the frame as described above.

Further details regarding transcatheter prosthetic heart valves including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. Publication No. 2018/0325665, and U.S. Provisional Application No. 62/854,702, filed May 30, 2019, all of which are incorporated herein by reference in their entireties.

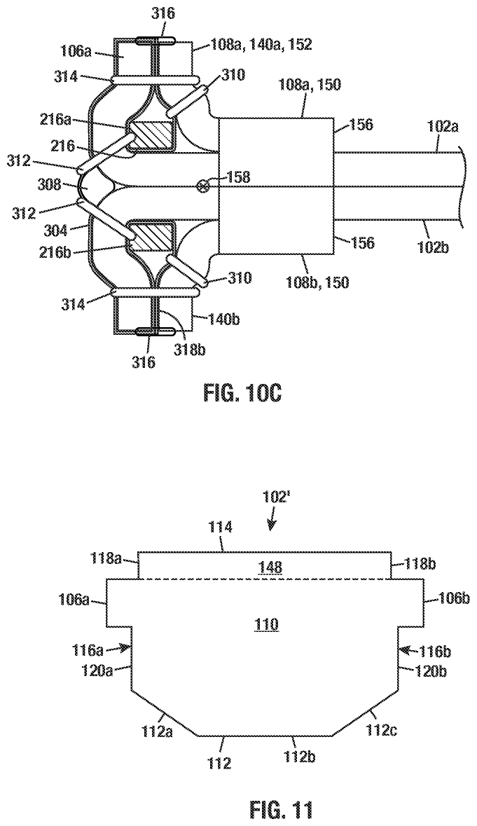

FIG. 11 shows an alternative leaflet 102′ that can be incorporated in any of the prosthetic valves disclosed herein. For example, in one implementation, the prosthetic valve 300 can include three leaflets 102′. The leaflet 102′ is similar to the leaflet 102 except that the former does not include the secondary tabs 108a, 108b. Thus, in this example, the leaflet 102′ does not have tab portions that overlap the upper material portion 148, but the free edge 114 nonetheless is offset in a downstream direction from the commissures (formed by primary tabs 106a, 106b) by the side edges 118a, 118b and the upper material portion 148. The side edges 118a, 118b are not attached to the frame and therefore allow the upper material portions 148 to provide slack portions that can be tensioned during valve closure, allowing the free edges of the leaflets to move closer together and promote coaptation of the leaflets.

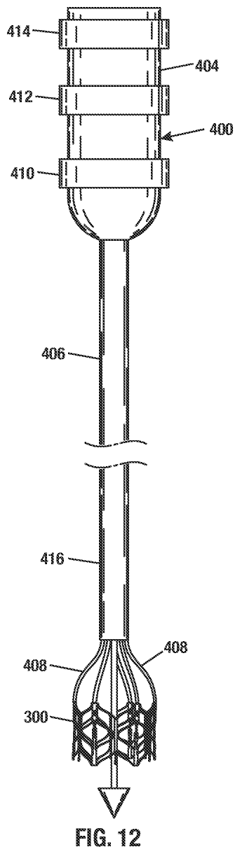

FIG. 12 shows an exemplary delivery device 400 that can be used to deliver the prosthetic heart valve 300 to an implantation site. The delivery device 400 generally includes a handle 404, an elongated shaft 406 extending distally from handle 404, and one or more actuation assemblies 408 extending distally through the elongated shaft 406. The actuator assemblies 408 are configured to radially expand and/or radially collapse the prosthetic valve 300 when actuated as well as form a releasable connection with the prosthetic valve. A distal end portion 416 of the shaft 406 can be sized to house the prosthetic valve 300 in a radially compressed state while traveling through the patient's vasculature. In this manner, the distal end portion 416 functions as a delivery sheath or capsule for delivery of the prosthetic valve.

The actuator assemblies 408 can be releasably coupled to the prosthetic valve 300. In one example, the actuator assemblies 408 can be coupled to the actuators of the prosthetic valve. The actuator assemblies 408 may be coupled to some or all of the actuators. For example, the frame of the prosthetic valve shown in FIGS. 8A and 8B has six actuator portions (228 in FIG. 8B). In this case, the delivery device 400 may include up to six actuator assemblies 408 to engage the actuator portions 228 of the prosthetic valve. Each actuator assembly 408 can include a first actuation member configured as a support tube and a second actuation configured as a driver. The driver can extend through the support tube and can have a distal end portion that engages the head 244 of the threaded rod 240 of the actuator portion 228 shown in FIG. 8B). The support tube has a distal end portion that can abut an adjacent section of the frame 200. The driver and the support tube can cooperate to form a releasable connection with the threaded rod 240.

The handle 404 can include one or more control mechanisms (for example, knobs or other actuating mechanisms) for controlling different components of the delivery device 400 in order to expand and/or deploy the prosthetic valve 300. For example, the handle 404 may include first, second, and third knobs 410, 412, and 414.

In one example, the first knob 410 can be a rotatable knob configured to produce axial movement of the shaft 406 relative to prosthetic valve 300 in the distal and/or proximal directions in order to deploy the prosthetic valve from the delivery sheath 416 once the prosthetic valve has been advanced to a location at or adjacent the desired implantation location within the patient's body. For example, rotation of the first knob 410 in a first direction (for example, clockwise) can retract the sheath 416 proximally relative to the prosthetic valve 300 and rotation of the first knob 410 in a second direction (for example, counterclockwise) can advance the sheath 416 distally. In other examples, the first knob 410 can be actuated by sliding or moving the knob 410 axially, such as pulling and/or pushing the knob. In other examples, actuation of the first knob 410 (rotation or sliding movement of the knob 410) can produce axial movement of the actuator assemblies 408 (and therefore the prosthetic valve 300) relative to the delivery sheath 416 to advance the prosthetic valve distally from the sheath 416.

The second knob 412 can be a rotatable knob configured to produce radial expansion and/or contraction of the prosthetic valve 300. For example, the proximal end portions of the drivers can be operatively coupled to the second knob 412 such that rotation of the second knob 412 can rotate the drivers relative to the support tubes and produce corresponding rotation of the threaded rods 240 of the prosthetic valve. Rotation of the second knob 412 in a first direction (for example, clockwise) can radially expand the prosthetic valve 300, and rotation of the second knob 412 in a second direction (for example, counterclockwise) can radially collapse the prosthetic valve 300. In other examples, for example, if the actuator portions 228 of the prosthetic valve are reciprocating type actuators, the second knob 412 can be actuated by sliding or moving second knob 412 axially, such as pulling and/or pushing the knob.

The third knob 414 can be a rotatable knob configured to release the prosthetic valve 300 from the delivery device 400. For example, the proximal end portions of the support tubes can be operatively coupled to the third knob 414 such that rotation of the third knob 414 produces axial movement of the support tubes relative to the drivers. Rotation of the third knob in a first direction (for example, clockwise) can move the support tubes in a proximal direction relative to the drivers and away from the prosthetic valve. Once the distal end portions of the support tubes are retracted proximally beyond the distal end portions of the drivers that engage the threaded rods 240, the distal end portions of the drivers can be configured to automatically disengage from the threaded rods 240. In other examples, the third knob 414 can be actuated by sliding or moving the third knob 414 axially, such as pulling and/or pushing the knob. Further details regarding the delivery device 400 are disclosed in U.S. Application No. 63/085,947, filed Sep. 30, 2020, which is incorporated herein by reference. Other delivery devices can be used to deliver and implant the prosthetic valves disclosed herein, such as those disclosed in PCT Application No. PCT/US2020/063104, filed Dec. 3, 2020, and U.S. Application No. 62/990,299, filed Mar. 16, 2020, which are incorporated herein by reference.

A method of delivering a prosthetic valve 300 generally includes placing the prosthetic valve 300 in a radially compressed state, for example, by operating the actuator portions 228 integrated with the frame of the prosthetic valve to place the frame in a radially compressed configuration. This can be accomplished by releasably coupling the prosthetic valve to the actuator assemblies 408 of the delivery device and actuating the second knob 412. The radially compressed prosthetic valve optionally can be placed within a sheath 416 of the delivery device. The delivery device and prosthetic valve can be advanced over a guidewire through the vasculature of a patient to a selected implantation site (for example, the native aortic annulus). For example, when implanting the prosthetic valve within the native aortic valve, the delivery device and prosthetic valve can be inserted into and through a femoral artery, and through the aorta to the native aortic valve. At the implantation site, if initially contained within the sheath 416, the prosthetic valve 300 can be deployed from the sheath 416 by actuating the first knob 410. The prosthetic valve 300 can then be radially expanded to a desired size by actuating the second knob 412. Once the prosthetic device is at the desired diameter, the actuator assemblies of the delivery device are uncoupled from the prosthetic valve by actuating the third knob 414, allowing removal of the delivery device from the patient's body.

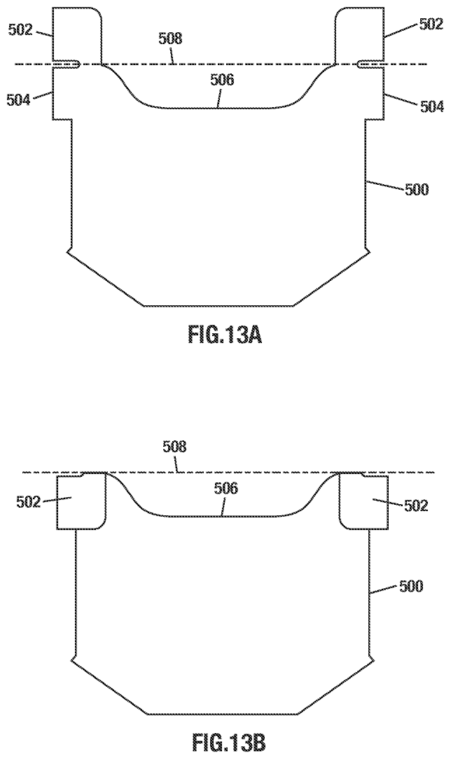

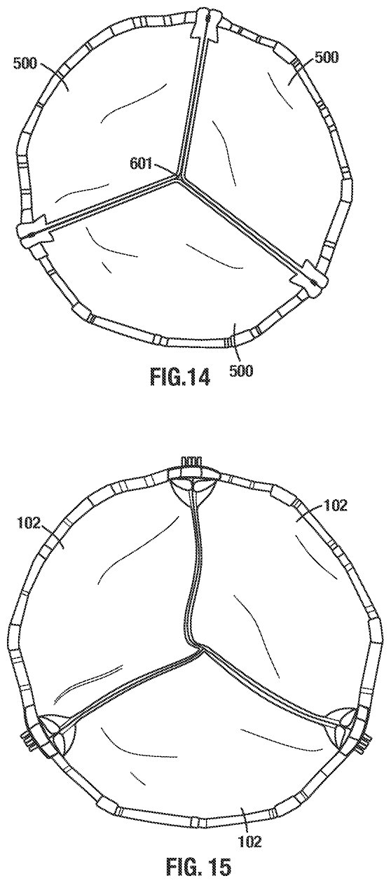

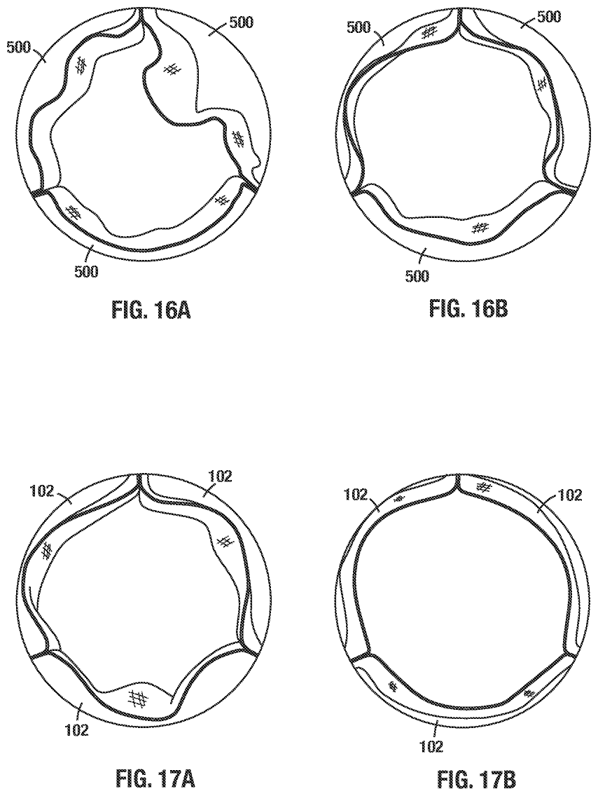

Once the prosthetic valve 300 is implanted, the leaflets of the valve can cycle between closed and open states during the diastolic and systolic phases. When the leaflets 102 are used as the leaflets of the valve, the folded material portions of the leaflets take part in closing of the valve and contribute to the ability of the leaflets to coapt completely. Examples A-F below compare the leaflets with folded material portions as in FIG. 3 with an alternative leaflet design.
EXAMPLE A (COMPARATIVE): A prosthetic valve was constructed using three leaflets having an alternative leaflet 500 as shown in FIG. 13A. The leaflet 500 has upper tabs 502, lower tabs 504, a leaflet free edge 506, and a fold line 508 about which upper tabs 502 fold. The fold line 508 is aligned or substantially aligned with the upper edges of the lower tabs 504 and is at a higher level compared to the leaflet free edge 506 (the fold line 508 is axially offset from the free edge 506 in the downstream direction). FIG. 13B shows the upper tabs 502 folded down about the fold line 508. The construction of the prosthetic valve was generally as described for prosthetic valve 300, except that the leaflets having the design shown in FIG. 13A are used instead of the leaflets having the design shown in FIG. 3. In this example, the commissures formed at the location of the lower tabs 504 are at the same level as the fold line 508. The leaflets 500 do not have folded material portions that protrude relative to commissures formed at the location of lower tabs 504.
EXAMPLE B: A prosthetic valve was constructed using three leaflets 102 having the leaflet design shown in FIG. 3. The construction of the prosthetic valve was generally as described for prosthetic valve 300. The upper material portions of the leaflets in the valve had a height of 3 mm in the open state.
EXAMPLE C (COMPARATIVE): A prosthetic valve using the leaflets 500 was constructed as in comparative Example A with a valve diameter of 29 mm. FIG. 14 shows an illustration of a frame of a video recording made as the leaflets transitioned between open and closed states. In the video frame illustrated in FIG. 14, the leaflets 500 are in a closed state. As shown, the leaflets fail to establish complete coaptation with each other in the closed state, leaving a small central opening 601 therebetween that may result in backflow.
EXAMPLE D: A prosthetic valve was constructed using the leaflets 102 with folded material portions as described in Example B with a valve diameter of 29 mm. FIG. 15 shows an illustration of a frame of a video recording made as the leaflets transitioned between open and closed states. In the video frame illustrated in FIG. 15, the leaflets are in a closed state. As shown in FIG. 15, the leaflets 102 establish complete proper coaptation with each other. The upper material portions of the leaflets 102 downstream of the commissures are pulled radially inwardly and contribute material slack that allow the leaflets to extend further toward each other and fully coapt with each other. As shown, the additional slack material causes the leaflets to form small circumferential folds at the center of coaptation.
EXAMPLE E (COMPARATIVE): Prosthetic valves using the leaflets 500 were constructed as described in comparative Example A with valve diameters of 26 mm and 29 mm. FIG. 16A shows the leaflets 500 in an open state for the valve diameter of 26 mm. FIG. 16B shows the leaflets 500 in an open state for the valve diameter of 29 mm. As shown, the leaflets 500 can form significant ripples and folds in the open state, which can cause flow interference and increased pressure gradients across the valve, especially for the smaller valve diameter of 26 mm.
EXAMPLE F: Prosthetic valves using the leaflets 102 with folded material portions were constructed as described in Example B with valve diameters of 26 mm and 29 mm. FIG. 17A shows the leaflets 102 with the upper material portions in an open state for the valve diameter of 26 mm. FIG. 17A shows the leaflets 102 with the upper material portions in an open state for the valve diameter of 29 mm. Compared to the valve openings in FIGS. 16A and 16B and comparative Example E, the valve openings in FIGS. 17A and 17B have a larger opening with less folds and ripples.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

Additional examples based on principles described herein are enumerated below.
Example 1: A prosthetic valve comprising: an annular frame and a plurality of leaflets positioned within the annular frame and attached to the annular frame at a plurality of locations on the annular frame. Each of the leaflets comprising: a flexible sheet having a leaflet attachment edge, a leaflet free edge in opposing relation to the leaflet attachment edge, opposite first and second sides extending in an axial direction between the leaflet attachment edge and the leaflet free edge, and an upper material portion of a select height connected to the leaflet free edge; a first primary tab and a second primary tab projecting respectively from the opposite first and second sides, the first and second primary tabs offset from the leaflet free edge by the upper material portion; and a first secondary tab and a second secondary tab connected respectively to opposite ends of the leaflet free edge, the first and second secondary tabs folded about a fold line and over the upper material portion, wherein the upper material portion contributes a material slack that extends a radial reach of the leaflet free edge for coaptation.
Example 2: The prosthetic valve of any example herein, particularly example 1, wherein the select height is greater than a thickness of the flexible sheet.
Example 3: The prosthetic valve of any example herein, particularly any one of examples 1 and 2, wherein the select height is in a range from 0.5 mm to 5 mm.
Example 4: The prosthetic valve of any example herein, particularly any one of examples 1 and 2, wherein the select height is in a range from 0.5 mm to 2 mm.
Example 5: The prosthetic valve of any example herein, particularly any one of examples 1 to 4, wherein the fold line is aligned with the leaflet free edge.
Example 6: The prosthetic valve of any example herein, particularly any one of examples 1 to 5, wherein each of the first and second secondary tabs has a first tab portion and a second tab portion, and wherein the first and second secondary tabs are folded about the fold line to unite the first tab portions respectively with the first and second primary tabs and to overlap the second tab portions respectively with the upper material portion of the flexible sheet.
Example 7: The prosthetic valve of any example herein, particularly any one of examples 1 to 6, wherein the second tab portion of each of the first and second secondary tabs is offset from the leaflet free edge by a height of the respective first tab portion.
Example 8: The prosthetic valve of any example herein, particularly any one of example 6 and 7, wherein the first tab portion of each of the first and second secondary tabs and the second tab portion of each of the first and second secondary tabs are arranged orthogonally to form an L shape.
Example 9: The prosthetic valve of any example herein, particularly any one of examples 6 to 8, wherein the first tab portions of the first and second secondary tabs are connected respectively to the opposite ends of the leaflet free edge.
Example 10: The prosthetic valve of any example herein, particularly any one of examples 1 to 9, wherein each one of the opposite first and second sides of the flexible sheet includes a first side edge and a second side edge spaced apart in the axial direction, and wherein the first and second primary tabs projecting from the opposite first and second sides of the flexible sheet extend between the respective first side edge and second side edge.
Example 11: The prosthetic valve of any example herein, particularly example 10, wherein each of the first side edges of the opposite first and second sides is connected to one of the first and second secondary tabs, and wherein the second side edges of the opposite first and second sides are connected respectively to opposite ends of the leaflet attachment edge.
Example 12: The prosthetic valve of any example herein, particularly example 11, wherein the second side edges of adjacent leaflets are attached together.
Example 13: The prosthetic valve of any example herein, particularly any one of examples 1 to 12, wherein the leaflet attachment edge forms a truncated V-shape.
Example 14: The prosthetic valve of any example herein, particularly any one of examples 1 to 13, further comprising at least one skirt disposed around the annular frame as a sealing member.
Example 15: The prosthetic valve of any example herein, particularly any one of examples 1 to 14, wherein the annular frame comprises a plurality of curved struts arranged to define a plurality of columns of cells.
Example 16: The prosthetic valve of any example herein, particularly example 15, further comprising at least one actuator portion coupled to at least a portion of the curved struts and operable to deflect the at least a portion of the curved struts to radially expand or radially compress the annular frame.
Example 17: The prosthetic valve of any example herein, particularly any one of examples 15 and 16, wherein the plurality of curved struts are divided into three frame sections, and wherein the annular frame comprises three frame posts interconnecting the three frame sections.
Example 18: The prosthetic valve of any example herein, particularly example 17, wherein at least one opening is formed on each of the three frame posts to receive the first primary tabs of two adjacent leaflets.
Example 19: A prosthetic valve comprising: an annular frame having an inflow end, an outflow end, a longitudinal axis defining an axial direction, and a plurality of commissure nodes positioned along a circumference of the annular frame; and a valvular structure mounted within the annular frame, the valvular structure comprising a plurality of leaflets and a plurality of commissures that attach the plurality of leaflets to the annular frame at the plurality of commissure nodes. Each leaflet comprising: a leaflet attachment edge positioned in a portion of the annular frame including the inflow end and attached to the annular frame; a leaflet free edge positioned in a portion of the annular frame including the outflow end; and an upper material portion of a select height connected to the leaflet free edge, wherein the upper material portion offsets the leaflet free edge from the plurality of commissures in an axial direction by the select height, wherein the upper material portion contributes a material slack that extends a radial reach of the leaflet free edge for coaptation.
Example 20: The prosthetic valve of any example herein, particularly example 19, wherein each leaflet comprises a first primary tab and a second primary tab projecting respectively from opposite sides of the leaflet and a first secondary tab and a second secondary tab connected respectively to the opposite ends of the leaflet free edge, each of the first and second secondary tabs respectively forming a cooperating pair with the first and second primary tabs.
Example 21: The prosthetic valve of any example herein, particularly example 20, wherein the first and second primary tabs and the first and second secondary tabs of the plurality of leaflets form the plurality of commissures at the plurality of commissure nodes.
Example 22: The prosthetic valve of any example herein, particularly any one of examples 20 and 21, wherein the first and second secondary tabs of each leaflet are folded about a fold line of the leaflet into a cooperating position respectively with the first and second primary tabs.
Example 23: The prosthetic valve of any example herein, particularly example 22, wherein the fold line of each leaflet is aligned with the leaflet free edge of the leaflet.
Example 24: The prosthetic valve of any example herein, particularly any one of examples 19 to 23, wherein each leaflet comprises a flexible sheet having a thickness, and wherein the select height is greater than the thickness.
Example 25: The prosthetic valve of any example herein, particularly any one of examples 19 to 23, wherein the select height is in a range from 0.5 mm to 5 mm.
Example 26: The prosthetic valve of any example herein, particularly any one of examples 19 to 25, wherein the annular frame comprises a plurality of frame sections corresponding to the plurality of leaflets, each of the frame sections comprising a plurality of interconnected struts.
Example 27: The prosthetic valve of any example herein, particularly example 26, wherein the leaflet attachment edges of the plurality of leaflets are attached to a portion of the plurality of interconnected struts adjacent to the inflow end of the annular frame.
Example 28: The prosthetic valve of any example herein, particularly any one of examples 26 and 27, wherein each strut is curved.
Example 29: The prosthetic valve of any example herein, particularly example 28, wherein a first portion of the plurality of interconnected struts forms a first curved geometry, and wherein a second portion of the plurality of interconnected struts forms a second curved geometry that is inverted relative to the first curved geometry.
Example 30: The prosthetic valve of any example herein, particularly any one of examples 26 to 29, wherein the plurality of frame sections are interconnected by a plurality of main frame posts, and wherein the plurality of commissure nodes are formed on the main frame posts.
Example 31: The prosthetic valve of any example herein, particularly example 30, wherein each frame section comprises a first frame subsection and a second frame subsection interconnected by an auxiliary frame post, each of the first frame subsection and the second frame subsection comprising a portion of the plurality of interconnected struts.
Example 32: The prosthetic valve of any example herein, particularly example 31, further comprising a plurality of cantilevered struts, each of the plurality of cantilevered struts attached to one of the auxiliary frame posts and main frame posts.
Example 33: The prosthetic valve of any example herein, particularly any one of examples 31 and 32, wherein the leaflet attachment edges of the plurality of leaflets are attached to the plurality of cantilevered struts.
Example 34: The prosthetic valve of any example herein, particularly any one of examples 32 and 33, wherein the cantilevered struts are biased to expand from a first position parallel to the axial position to a second position angled radially outward relative to the axial direction, wherein the prosthetic valve further comprises an outer skirt mounted to the cantilevered struts.
Example 35: The prosthetic valve of any example herein, particularly any one of examples 19 to 34, wherein the plurality of commissure nodes are offset in the axial direction relative to the outflow end of the annular frame.
Example 36: The prosthetic valve of any example herein, particularly example 35, wherein the plurality of commissure nodes are offset in the axial direction by an offset distance in a range from 2 mm to 6 mm.
Example 37: The prosthetic valve of any example herein, particularly any one of examples 35 and 36, wherein the first and second folded material portions are positioned in a portion of the annular frame between the commissure nodes and the outflow end.
Example 38: The prosthetic valve of any example herein, particularly any one of examples 26 to 37, wherein each frame section comprises at least one actuator portion coupled to the plurality of interconnected struts in the frame section, and wherein the at least one actuator portion is operable to radially expand or compress the annular frame by deflecting the plurality of interconnect struts.
Example 39: The prosthetic valve of any example herein, particularly example 38, wherein the at least one actuator portion comprises a pair of support arms spaced apart in the axial direction and a threaded rod engaged with the pair of support arms and rotatable to adjust a gap between the pair of support arms.
Example 40: The prosthetic valve of any example herein, particularly any one of examples 19 to 39, further comprising at least one skirt disposed around the annular frame as a sealing member.
Example 41: A delivery apparatus comprising: a delivery device comprising a handle; and a prosthetic valve releasably coupled to the delivery device, the prosthetic valve comprising an annular frame and a valvular structure mounted within the annular frame, the valvular structure comprising a plurality of leaflets defining a plurality of commissures coupled to the annular frame, each leaflet comprising a free edge that is offset in a downstream direction from the commissures by sides edges that are not attached to the frame.
Example 42: The delivery apparatus of any example herein, particularly example 41, wherein the annular frame comprises at least one actuator portion that is operable to radially expand or compress the annular frame.
Example 43: The delivery apparatus of any example herein, particularly example 42, wherein the delivery device further comprises an elongated shaft extending distally from the handle and at least one actuator assembly extending distally through the elongated shaft, and wherein the at least one actuator assembly is releasably engaged with the at least one actuator portion.
Example 44: The delivery apparatus of any example herein, particularly any of examples 41 to 43, wherein each leaflet comprises an upper material portion extending between the side edges that are not attached to the frame and protruding relative to the plurality of commissures in the downstream direction by a select height, the upper material portion to contribute a material slack that extends a radial reach of the free edge for coaptation.
Example 45: The delivery apparatus of any example herein, particularly example 44, wherein each leaflet comprises a first primary tab and a second primary tab projecting respectively from opposite sides of the leaflet and a first secondary tab and a second secondary tab connected respectively to opposite ends of the free edge of the leaflet, and wherein the first and second primary tabs and the first and second secondary tabs of the plurality of leaflets form the plurality of commissures.
Example 46: The delivery apparatus of any example herein, particularly example 45, wherein the first and second secondary tabs of each leaflet are folded about a fold line that is aligned with the free edge of the leaflet.
Example 47: The delivery apparatus of any example herein, particularly any one of examples 44 to 46, wherein the select height is in a range from 0.5 mm to 5 mm.
Example 48: The delivery apparatus of any example herein, particularly any one of examples 44 to 47, wherein the annular frame has an inflow end, an outflow end, and a longitudinal axis extending from the inflow end to the outflow end and defining an axial direction, wherein the plurality of commissures are offset from the outflow end in the axial direction, and wherein the upper material portion of each leaflet is positioned in a portion of the annular frame between the plurality of commissures and the outflow end.
Example 49: A method comprising: inserting a distal end of the delivery apparatus of any one of examples 41 to 48 into a vasculature of a patient; advancing the prosthetic valve at the distal end to a selected implantation site; actuating the handle to release the prosthetic valve from the delivery device; and withdrawing the delivery device from the patient with the prosthetic valve implanted at the selected implantation site.
Example 50: The method of any example herein, particularly example 49, further comprising radially compressing the prosthetic valve prior to inserting the distal end of the delivery apparatus into the vasculature of the patient.
Example 51: The method of any example herein, particularly any one of examples 49 and 50, further comprising actuating the handle to cause radial expansion of the prosthetic valve at the selected implantation site.
Example 52: A prosthetic valve comprising: an annular frame; and a valvular structure mounted within the annular frame, the valvular structure comprising a plurality of leaflets defining a plurality of commissures coupled to the annular frame, each leaflet comprising a leaflet free edge that is offset in a downstream direction from the commissures by side edges that are not attached to the frame, wherein the side edges contribute a material slack respectively at the opposite ends of the leaflet free edge that extends a radial reach of the leaflet free edge for coaptation.
Example 53: The prosthetic valve of any example herein, particularly example 52, wherein each leaflet comprises: a first primary tab and a second primary tab on sides of the leaflet, wherein the first and second primary tabs are offset from the leaflet free edge by the side edges that are not attached to the frame, wherein each primary tab is paired with an adjacent primary tab of an adjacent leaflet to form one of the commissures.
Example 54: The prosthetic valve of any example herein, particularly example 53, wherein each leaflet further comprises a first secondary tab and a second secondary tab connected respectively to the opposite ends of the leaflet free edge, the first and second secondary tabs folded about a fold line to position the first and secondary tabs in a cooperating relation with the first and second primary tabs.
Example 55: The prosthetic valve of any example herein, particularly any one of examples 52 to 54, wherein a height of the side edges that are not attached to the frame is in a range from 0.5 mm to 5 mm.
Example 56: The prosthetic valve of any example herein, particularly any one of examples 52 to 55, further comprising at least one skirt disposed around the annular frame as a sealing member.
Example 57: The prosthetic valve of any example herein, particularly any one of examples 52 to 56, wherein the annular frame comprises a plurality of curved struts arranged to define a plurality of columns of cells.
Example 58: The prosthetic valve of any example herein, particularly example 57, wherein the annular frame further comprises at least one actuator portion coupled to at least a portion of the curved struts and operable to deflect the at least a portion of the curved struts to radially expand or radially compress the annular frame.
Example 59: The prosthetic valve of any example herein, particularly any one of examples 52 to 58, wherein the leaflets are configured to move between an open state and a close state to regulate the flow of blood through the prosthetic valve, wherein the side edges of the leaflets that are not attached to the frame are spaced radially inwardly from the frame when the leaflets are in the closed state.
Example 60: The prosthetic valve of any example herein, particularly any one of examples 52 to 59, wherein the frame comprises a plurality of cantilevered struts at an inflow end of the frame, wherein the plurality of cantilevered struts are biased to expand from a first position parallel to a longitudinal axis of the frame to a second position angled radially outward relative to the longitudinal axis, wherein the prosthetic valve further comprises an outer skirt mounted to the plurality of cantilevered struts, and wherein the plurality of cantilevered struts are configured to urge the outer skirt against surrounding tissue when the prosthetic valve is deployed at an implantation site and the plurality of cantilevered struts move to the second position.
Example 61: The prosthetic valve of any example herein, particularly any one of examples 53 to 54, wherein each leaflet comprises a leaflet attachment edge in opposing relation to the leaflet free edge, wherein the leaflet attachment edge is connected to the frame.
Example 62: The prosthetic valve of any example herein, particularly example 61, wherein the leaflet attachment edge forms a truncated V-shape.
Example 63: The prosthetic valve of any example herein, particularly any one of examples 61 to 62, wherein each leaflet comprises first and second opposing side edges extending axially between the leaflet attachment edge and the first and second primary tabs.
Example 64: The prosthetic valve of any example herein, particularly any one of examples 52 to 60, wherein the prosthetic valve is sterilized.

In view of the many possible examples to which the principles of the disclosed invention may be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

1. A prosthetic valve comprising:
an annular frame; and
a valvular structure mounted within the annular frame, the valvular structure comprising a plurality of leaflets defining a plurality of commissures coupled to the annular frame, each leaflet comprising a leaflet free edge that is offset in a downstream direction from the commissures by side edges that are not attached to the annular frame, wherein the side edges contribute a material slack respectively at the opposite ends of the leaflet free edge that extends a radial reach of the leaflet free edge for coaptation.

2. The prosthetic valve of claim 1, wherein each leaflet comprises:
a first primary tab and a second primary tab on sides of the leaflet, wherein the first and second primary tabs are offset from the leaflet free edge by the side edges that are not attached to the annular frame, wherein each primary tab is paired with an adjacent primary tab of an adjacent leaflet to form one of the commissures.

3. The prosthetic valve of claim 2, wherein each leaflet further comprises a first secondary tab and a second secondary tab connected respectively to the opposite ends of the leaflet free edge, the first and second secondary tabs folded about a fold line to position the first and secondary tabs in a cooperating relation with the first and second primary tabs.

4. The prosthetic valve of claim 2, wherein each leaflet comprises a leaflet attachment edge in opposing relation to the leaflet free edge, and wherein each leaflet comprises first and second opposing side edges extending axially between the leaflet attachment edge and the first and second primary tabs.

5. The prosthetic valve of claim 1, wherein a height of the side edges that are not attached to the annular frame is in a range from 0.5 mm to 5 mm.

6. The prosthetic valve of claim 1, further comprising at least one skirt disposed around the annular frame as a sealing member.

7. The prosthetic valve of claim 1, wherein the annular frame comprises a plurality of curved struts arranged to define a plurality of columns of cells.

8. The prosthetic valve of claim 7, wherein the annular frame further comprises at least one actuator portion coupled to at least a portion of the curved struts and operable to deflect the at least a portion of the curved struts to radially expand or radially compress the annular frame.

9. The prosthetic valve of claim 1, wherein the leaflets are configured to move between an open state and a close state to regulate the flow of blood through the prosthetic valve, and wherein the side edges of the leaflets that are not attached to the annular frame are spaced radially inwardly from the annular frame when the leaflets are in the closed state.

10. The prosthetic valve of claim 1, wherein the annular frame comprises a plurality of cantilevered struts at an inflow end of the annular frame, wherein the plurality of cantilevered struts are biased to expand from a first position parallel to a longitudinal axis of the annular frame to a second position angled radially outward relative to the longitudinal axis, wherein the prosthetic valve further comprises an outer skirt mounted to the plurality of cantilevered struts, and wherein the plurality of cantilevered struts are configured to urge the outer skirt against surrounding tissue when the prosthetic valve is deployed at an implantation site and the plurality of cantilevered struts move to the second position.

11. The prosthetic valve of claim 1, wherein each leaflet comprises a leaflet attachment edge in opposing relation to the leaflet free edge, and wherein the leaflet attachment edge is connected to the frame.

12. The prosthetic valve of claim 11, wherein the leaflet attachment edge forms a truncated V-shape.

13. A delivery apparatus comprising:
a delivery device comprising a handle; and
a prosthetic valve releasably coupled to the delivery device, the prosthetic valve comprising an annular frame and a valvular structure mounted within the annular frame, the valvular structure comprising a plurality of leaflets defining a plurality of commissures coupled to the annular frame, each leaflet comprising a free edge that is offset in a downstream direction from the commissures by side edges that are not attached to the annular frame.

14. The delivery apparatus of claim 13, wherein the annular frame comprises at least one actuator portion that is operable to radially expand or compress the annular frame.

15. The delivery apparatus of claim 13, wherein the delivery device further comprises an elongated shaft extending distally from the handle and at least one actuator assembly extending distally through the elongated shaft, and wherein the at least one actuator assembly is releasably engaged with the at least one actuator portion.

16. The delivery apparatus of claim 13, wherein each leaflet comprises an upper material portion extending between the side edges that are not attached to the annular frame and protruding relative to the plurality of commissures in the downstream direction by a select height, the upper material portion to contribute a material slack that extends a radial reach of the free edge for coaptation.

17. The delivery apparatus of claim 16, wherein each leaflet comprises a first primary tab and a second primary tab projecting respectively from opposite sides of the leaflet and a first secondary tab and a second secondary tab connected respectively to opposite ends of the free edge of the leaflet, and wherein the first and second primary tabs and the first and second secondary tabs of the plurality of leaflets form the plurality of commissures.

18. The delivery apparatus of claim 17, wherein the first and second secondary tabs of each leaflet are folded about a fold line that is aligned with the free edge of the leaflet.

19. The delivery apparatus of claim 16, wherein the select height is in a range from 0.5 mm to 5 mm.

20. The delivery apparatus of claim 16, wherein the annular frame has an inflow end, an outflow end, and a longitudinal axis extending from the inflow end to the outflow end and defining an axial direction, wherein the plurality of commissures are offset from the outflow end in the axial direction, and wherein the upper material portion of each leaflet is positioned in a portion of the annular frame between the plurality of commissures and the outflow end.

21. A method comprising:
inserting a distal end of a delivery device into a vasculature of a patient, wherein a prosthetic valve is releasably coupled to the distal end of the delivery device, wherein the prosthetic valve comprises an annular frame and a valvular structure mounted within the annular frame, wherein the valvular structure comprises a plurality of leaflets defining a plurality of commissures coupled to the annular frame, and wherein each leaflet comprises a free edge that is offset in a downstream direction from the commissures by side edges that are not attached to the annular frame;
advancing the prosthetic valve at the distal end of the delivery device to a selected implantation site;
actuating a handle of the delivery device to release the prosthetic valve from the distal end of the delivery device; and
withdrawing the delivery device from the patient with the prosthetic valve implanted at the selected implantation site.

22. The method of claim 21, further comprising:
radially compressing the prosthetic valve prior to inserting the distal end of the delivery device into the vasculature of the patient; and
actuating the handle of the delivery device to cause radial expansion of the prosthetic valve at the selected implantation site.
